(19) United States
(12) Patent Application Publication
(10) Pub. No.: US 2017/0156592 A1
(43) Pub. Date: Jun. 8, 2017

(54) HEALTHCARE SYSTEMS AND MONITORING METHOD FOR PHYSIOLOGICAL SIGNALS

(71) Applicant: MEDIATEK INC., Hsin-Chu (TW)

(72) Inventor: Chih-Ming FU, Hsinchu City (TW)

(21) Appl. No.: 15/354,126

(22) Filed: Nov. 17, 2016

Related U.S. Application Data
(60) Provisional application No. 62/261,900, filed on Dec. 2, 2015.

(51) Int. Cl. A61B 5/00; A61B 5/0464; A61B 5/0452; A61B 5/0205

(52) U.S. Cl. CPC A61B 5/0002; A61B 5/0022; A61B 5/02055; A61B 5/0464; A61B 5/0452; A61B 5/7264; A61B 5/742; A61B 5/02416

(57) ABSTRACT

A healthcare system is provided. The healthcare system includes a data server, an algorithm server, a display device, and a communication network. The data server stores a plurality of physiological signals. The algorithm server receives the plurality of physiological signals from the data server. The algorithm server applies a plurality of algorithms on the plurality of physiological signals to obtain at least one feature of the plurality of physiological signals and generates at least one label according to the at least one label. The display device displays the at least one label. The communication network communicatively connects the data server, the algorithm server, and the display device for providing signal transmission paths therebetween.

### CROSS REFERENCE TO RELATED APPLICATIONS

This application claims the benefit of U.S. Provisional Application No. 62/261,900, filed on Dec. 2, 2015, the contents of which are incorporated herein by reference.

### BACKGROUND OF THE INVENTION

Field of the Invention

The invention relates to a healthcare system, and more particularly to a healthcare system which can obtain labels for physiological signals.

Description of the Related Art

In some countries, such as India, 70% of the population lives in rural areas, but 3% of the total number of physicians in India practice there. Thus, a tele-health service was introduced to monitor the health of the people in the rural areas. The tele-health service is applied to obtain physiological signals from a patient (such as blood pressure, body temperature, heart rate, respiratory airflow and volume, oxygen saturation, and electrocardiography (ECG) signals) and transmits the physiological signals to a remote site for doctors through the network to make a diagnosis of a disease. The doctors may offer some feedback to the patient or local doctors for further treatment. Some physiological signals, such as ECG signals, need to be interpreted by cardiology specialists. However, there is a lack of cardiology specialists in India. If the ECG signals of all of the patients are transmitted to the cardiology specialists regardless of whether they suffer from cardiovascular diseases, the workload of the cardiology specialists will be very heavy and may result in inaccurate diagnosis of diseases.

### BRIEF SUMMARY OF THE INVENTION

An exemplary embodiment of a healthcare system, wherein the healthcare system comprises a data server, an algorithm server, a display device, and a communication network. The data server stores a plurality of physiological signals. The algorithm server receives the plurality of physiological signals from the data server. The algorithm server applies a plurality of algorithms on the plurality of physiological signals to obtain at least one feature of the plurality of physiological signals and generates a label according to the at least one feature. The display displays the label. The communication network communicatively connects the data server, the algorithm server, and the display device for providing signal transmission paths therebetween.

Another exemplary embodiment of a monitoring method comprises the steps of obtaining a plurality of physiological signals; applying a plurality of algorithms on the plurality of physiological signals to obtain at least one feature of the plurality of physiological signals; generating a label according to the at least one feature; and showing the label.

A detailed description is given in the following embodiments with reference to the accompanying drawings.

### BRIEF DESCRIPTION OF THE DRAWINGS

The invention can be more fully understood by reading the subsequent detailed description and examples with references made to the accompanying drawings, wherein:

FIG. 1 shows an exemplary embodiment of a healthcare system for physiological signals;

FIG. 2 is a schematic block diagram showing data transmission in the healthcare system of FIG. 1;

FIG. 3 shows a flow chart of an exemplary embodiment of a monitoring method for physiological signals;

FIG. 4 shows an exemplary embodiment of operations and algorithms of an algorithm server;

FIG. 5 shows an example of a flat line appearing on an ECG signal;

FIG. 6 shows an example of a sharp slop appearing on an ECG signal;

FIG. 7 shows an example of high-frequency appearing on an ECG signal;

FIG. 8 shows an example of a waveform of an ECG signal;

FIG. 9 shows an example of relationship between an ECG signal and a vessel pulse signal;

FIG. 10 shows an example of a heart axis;

FIG. 11A shows an example of a normal T-wave of an ECG signal;

FIG. 11B shows an example of a T-wave inversion of an ECG signal;

FIG. 12A shows an example of normal S and T-waves of an ECG signal;

FIG. 12B shows an example of an ST elevation of an ECG signal;

FIG. 13 shows an example of a change of R-R intervals of an ECG signal in a period of time; and

FIG. 14 shows an exemplary embodiment of a label list displayed on the display device of the healthcare system of FIG. 1.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

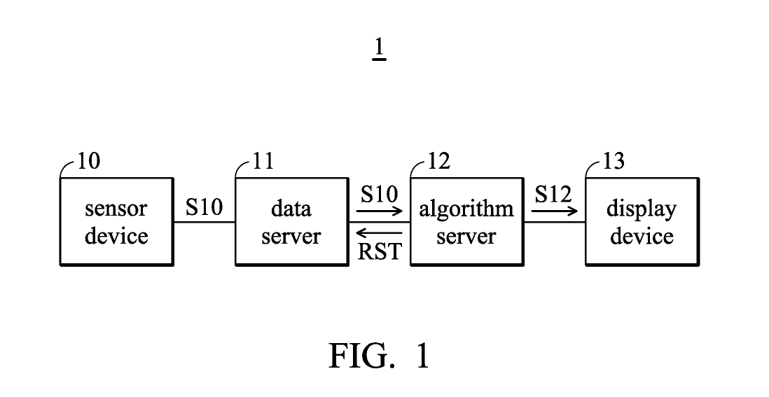

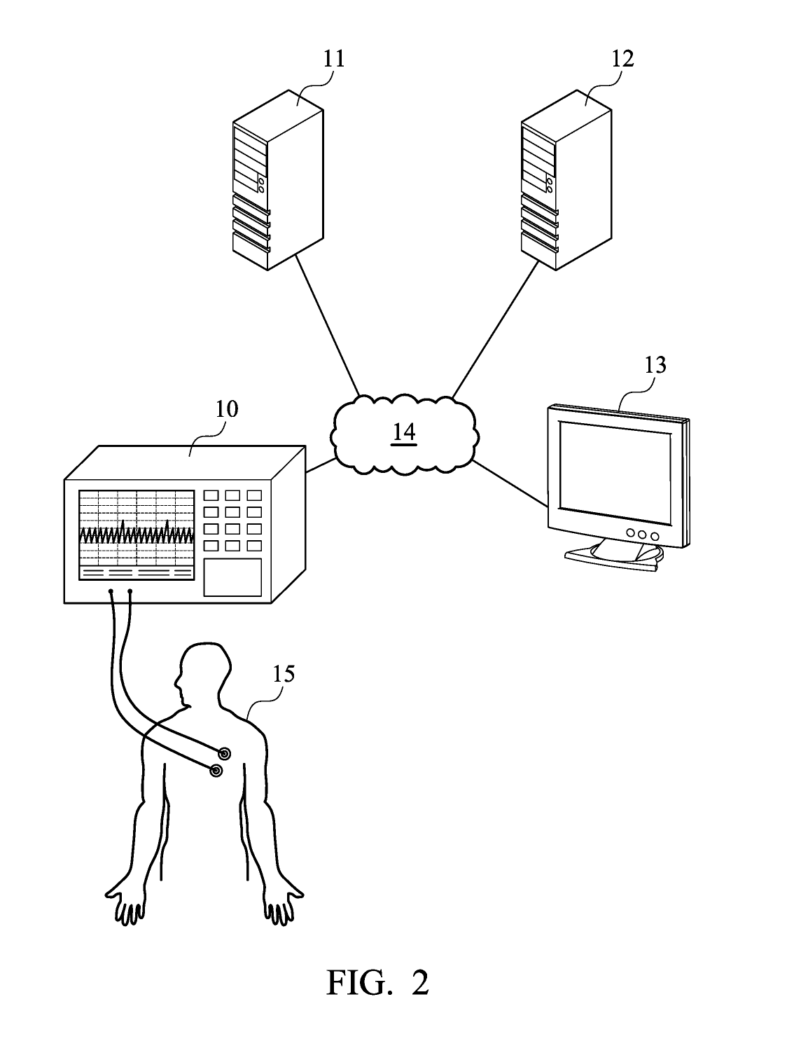

FIG. 1 shows an exemplary embodiment of a healthcare system for physiological signals. As shown in FIG. 1, a healthcare system 1 comprises a sensor device 10, a data server 11, an algorithm server 12, and a display device 13. The sensor device 10 detects at least one of the electrocardiography (ECG), photoplethysmogram (PPG), motion, body temperature, galvanic skin response, electroencephalograph, oxygen saturation, airflow in respiratory tract, heart rate, pulse wave transit time, and blood pressure of an object, such as a patient 15 shown in FIG. 2, and generates physiological signals S10 in response to the detection result. After obtaining the physiological signals S10, the sensor device 10 transmits the physiological signals S10 to the data server 11 through a communication network 14 shown in FIG. 2. The data server 11 receives the physiological signals S10 for storage. In the embodiment, the data server 11 can collect and store physiological signals from different objects, such as different patients. The algorithm server 12 can issue a request RST to read the physiological signals S10 stored in the data server 11 through the communication network 14. When the algorithm server 12 receives the physiological signals S10 of the patient 15 from the data server 11, the algorithm server 12 applies algorithms on the received physiological signals S10 to detect at least one feature of the physiological signals S10 and obtain at least one label for the physiological signals S10 according to the detected feature. In the following, one label is given as an example for illustration. The algorithm server 12 further generates a labeling result which comprises the information including the obtained label and detected feature. The display device 13 receives the labeling result S12 from the algorithm server 12 through the communication network 14 and displays a label list according to the labeling result S12. The label list includes the obtained label for the physiological signals S10. A viewer, such as a doctor, can know what label is given to the physiological signals S10.

In the embodiment, the label is classified into a not-screened-out category or a screened-out category. For example, the label may be an abnormal label, a normal label, or a noise label. An abnormal label is obtained for the physiological signals S10 through the applied algorithms when the patient 15 suffers from diseases. A normal label is obtained for the physiological signals S10 when the patient 15 does not suffer from diseases. A noise label is obtained for the physiological signals S10 when the quality of the physiological signals S10 is too low for a doctor to accept making a diagnosis of a disease.

A doctor, such as a general physician or a cardiology specialist, can be aware of what label is obtained for the physiological signals S10 according to the class of the features label and can thus make a decision to review the physiological signals S10 or not. In the embodiment, the abnormal label is classified into the not-screened-out category, while the normal label and the noise label are classified into the screened-out category. For example, when a doctor is aware of what would be considered an abnormal label obtained for the physiological signals S10, the doctor can retrieve the physiological signals S10 from the algorithm server 12 through the display device 13 in order to make a diagnosis of a disease.

In the embodiment, the communication network 15 is implemented by a tele-communication network, Internet, LAN, wireless LAN, or any combinations thereof to transmit signals or data between the sensor device 10, the data server 11, the algorithm server 12, and the display device 13. Any two of the tele-communication network, Internet, LAN and wireless LAN may be connected via gateways.

In the embodiment, the data server 11 can be implemented by dedicated-hardware that delivers database services or software executed by a processor, such as a general-purposed central processing unit (CPU), general-purposed graphics processing unit (GPU), micro-control unit (MCU), etc., for accomplishing the above operations. Similarly, the algorithm server 12 can be implemented by dedicated-hardware or software executed by a processor for accomplishing the above operations.

In an embodiment, the sensor device 10 also transmits patient information to the data server 11 for storage, such as the name and age of the patient 15. When the algorithm server 12 issues the request RST to the data server 11, the data server 11 transmits not only the physiological signals S10 but also the patient information to the algorithm server 12. Accordingly, the information of the labeling result S12 further comprises patient information, and the patient information can also be shown on the label list. In an embodiment, the labeling result S12 comprises a string with a JSON format.

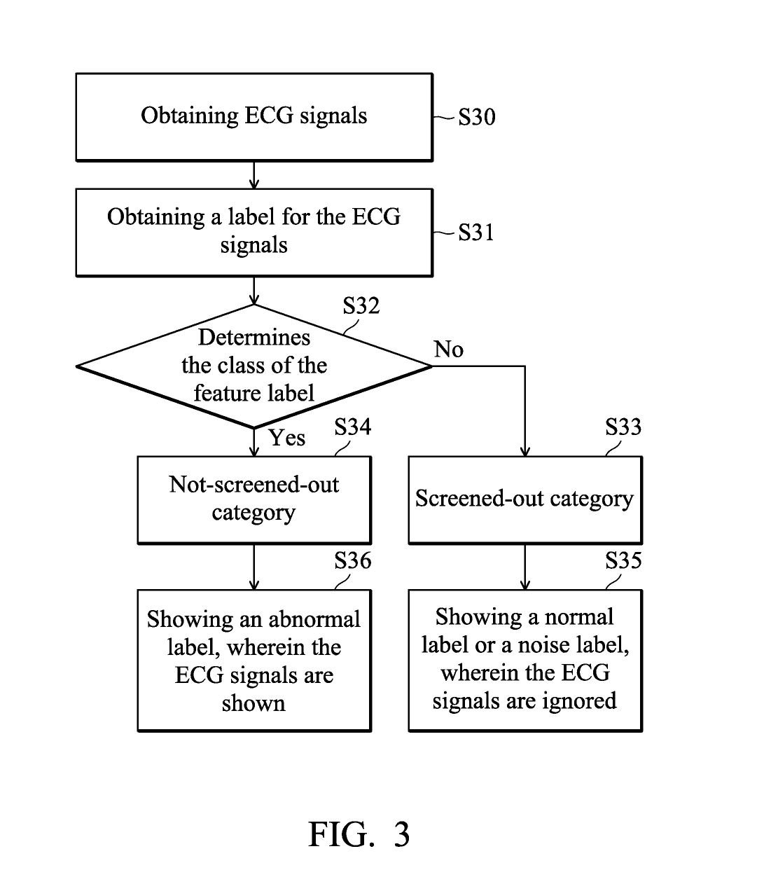

FIG. 3 shows a flow chart of an exemplary embodiment of a monitoring method for physiological signals. The monitoring method will be described by referring to FIG. 2 and FIG. 1. In the embodiment, electrocardiography (ECG) signals are given as an example for illustration. At the step S30, the sensor device 10 detects and obtains the ECG signals (physiological signals) of the patient 15. The ECG signals are stored in the data server 11. When receiving the ECG signals from the data server 11, the algorithm server 12 applies algorithms on the received ECG signals S10 to detect at least one feature of the ECG signals. The algorithm server 12 obtains a label for the ECG signals according to the detected feature (step S31). The algorithm server 12 generates the labeling result S12 to the display device 13. The display device 13 determines the class of the label (step S32). When the label is an abnormal label, it is classified into the not-screened-out category (step S34). The display device 13 displays a label list to show the abnormal label (step S36). As described above, the abnormal label indicates that the patient 15 may suffer from cardiovascular diseases. When a doctor, such as a general physician or a cardiology specialist, notes that there is an abnormal label on the label list which is classified into the not-screened-out category, the doctor may want to review the ECG signals to make a diagnosis of a disease. Thus, in cases where the doctor wants to review the ECG signals, the doctor issues a request through an input interference of the display device 15, and the display device 15 can retrieve the ECG signals from the algorithm server 12 in response to the request and show the ECG signals (step S36). When the label is a normal label or a noise label, it is classified into the screened-out category (step S33). The display device 13 shows the abnormal label on the label list (step S35). As described above, the normal label indicates that the patient 15 does not suffer from a disease and the noise label indicates that the quality of the ECG signals is too low. When the doctor notes that there is a normal label or a noise label on the label list which is classified into the screened-out category, the doctor is aware that the ECG signals can be ignored (step S35). Thus, the display device 15 does not need to retrieve the ECG signals from the algorithm server 12.

According to the above embodiment, since the label list comprises the label of the ECG signals, the doctor can determine whether the patient 15 may suffer from any cardiovascular diseases according to the label. Accordingly, the doctor may simply review the waveforms of the ECG signals whose label is classified into the not-screened-out category but ignores the ECG signals whose label is classified into the not-screened-out category, thereby reducing the workload. In another embodiment, if necessary, the doctor can also issues a request to review the waveforms of the ECG signals whose label is classified into the screened-out category.

In the above embodiment, one abnormal label is obtained for the physiological signals of the patient 15 who suffers from a disease. According to an embodiment, when the patient 15 suffers from diseases, several abnormal labels may be obtained for the physiological signals. Each abnormal label indicates one condition of a human body's organs. In the following, the detailed algorithms of the algorithm server 12 will be described by taking ECG signals as an example of the physiological signals. It has been known that ECG signals can represent the electrical activity of the human heart over a period of time by using ECG electrodes placed on the skin. For a conventional twelve-lead (12-lead) ECG, ten ECG electrodes are placed on the patient's limbs and on the surface of the chest. The overall magnitude of the heart's electrical potential is then measured from 12 different angles (“leads”) and is recorded over a period of time (usually several seconds). The twelve leads comprise I, II, III, aVL, aVR, aVF, V1, V2, V3, V4, V5, and V6, which serve as twelve ECG signals respectively.

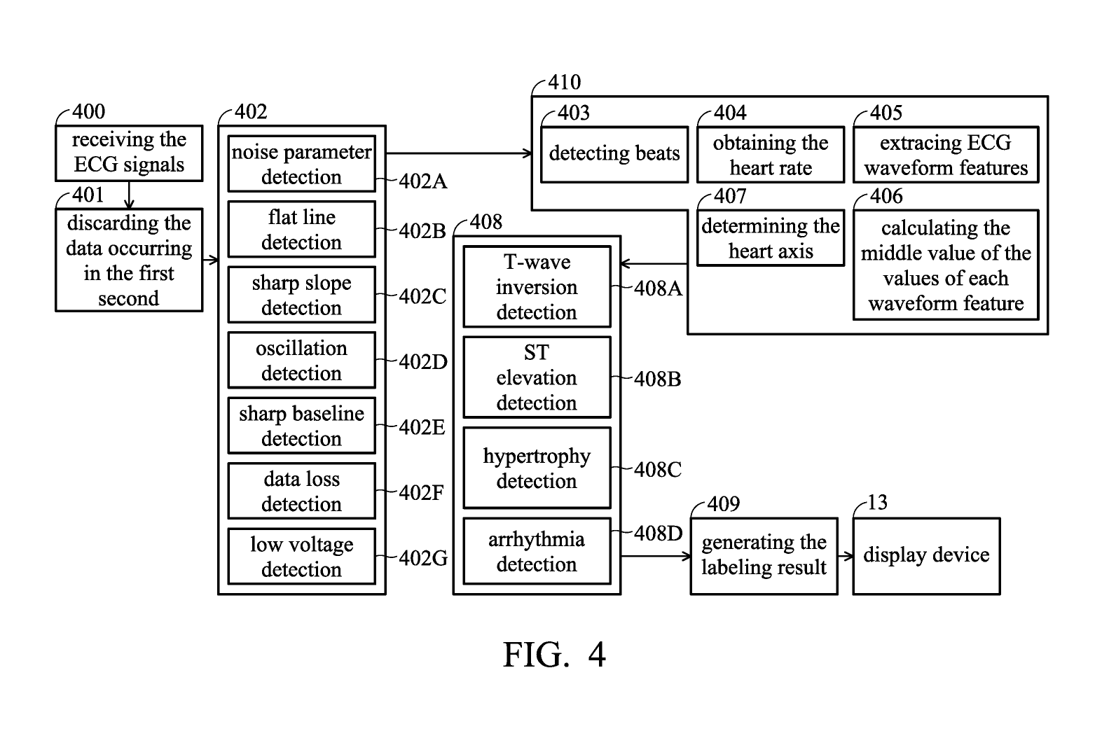

FIG. 4 shows an exemplary embodiment of the operations and algorithms of the algorithm server 12. The embodiment will be described by referring to FIGS. 2 and 4. Referring to FIG. 4, when the algorithm server 12 receives the ECG signals of the patient 15 from the data server 11 (block 400), the algorithm server 12 discards the data of each of the ECG signals occurring in the first second (block 401), and then performs a noise removal algorithm to remove the noise of each ECG signals (block 402). After the noise of each ECG signal is removed, the algorithm server 12 applies a quality estimation algorithm on each ECG signal (block 402).

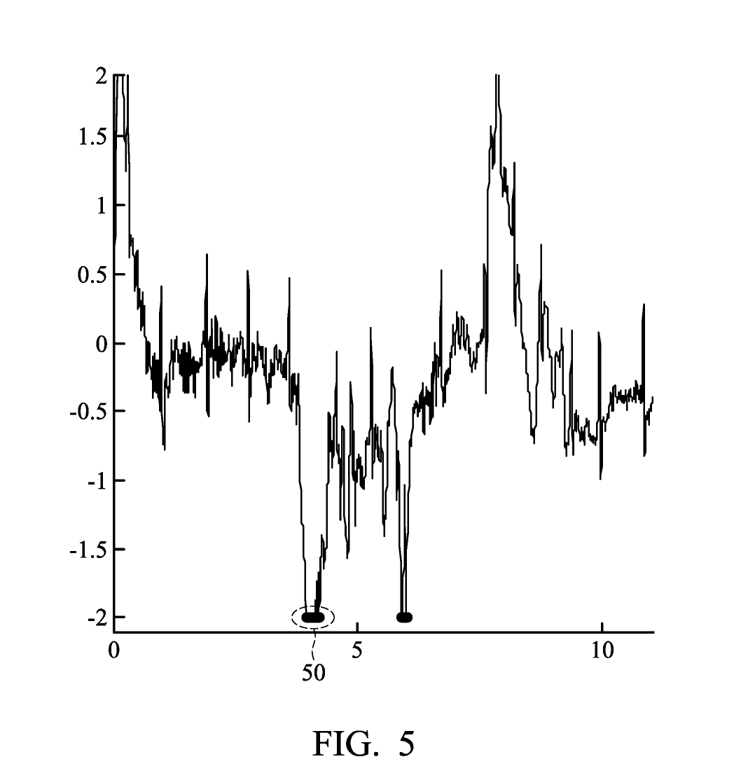

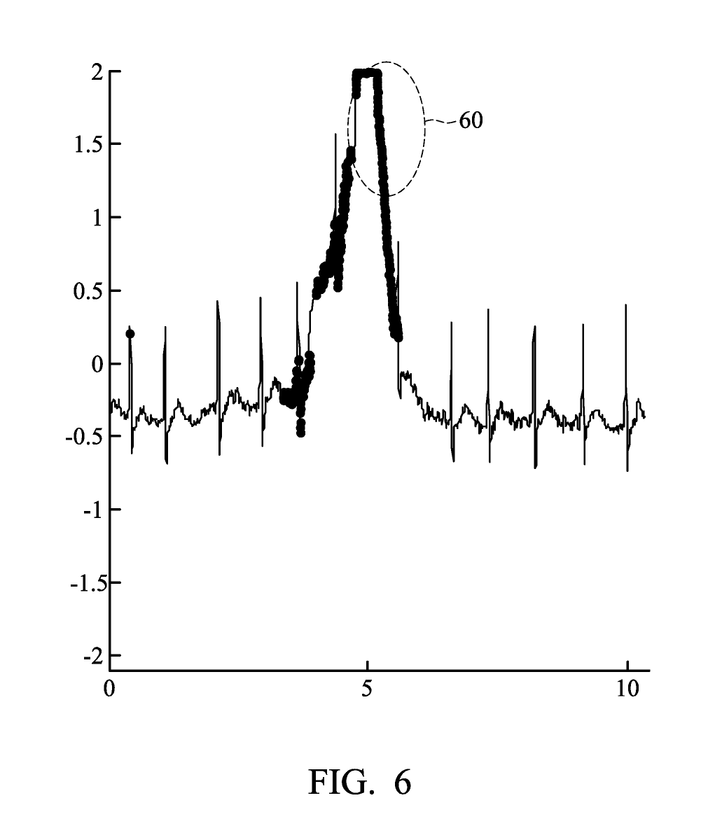

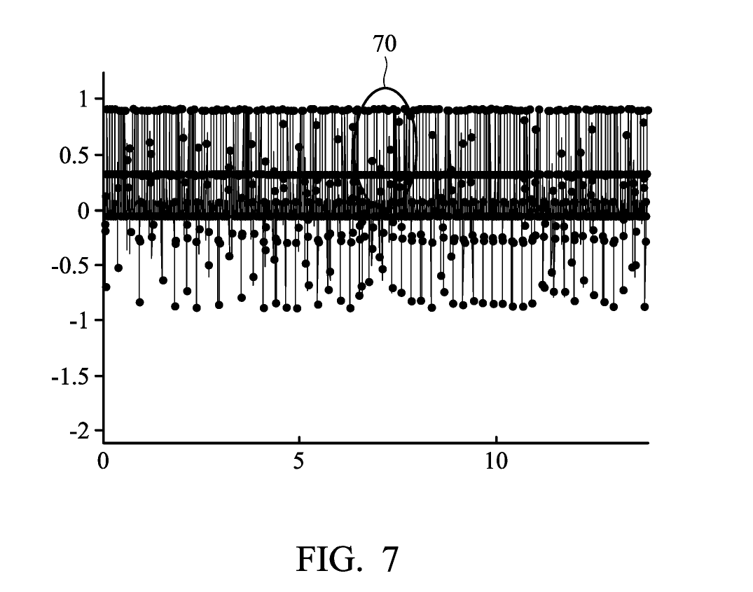

In an embodiment, when the quality estimation algorithm is applied on each ECG signal, the algorithm server 12 detects noise parameters of the ECG signal to estimate the quality of the ECG signal (block 402A). When the algorithm server 12 estimates that the quality of the ECG signal is low according to the noise parameters, the ECG signal is not trustworthy for diagnosing diseases. In another embodiment, when the quality estimation algorithm is applied on each ECG signal, the algorithm server 12 detects whether there is one flat line on the ECG signal or not (block 402B). Referring to FIG. 5, detecting a flat line 50 appearing on the ECG signal indicates that the corresponding ECG electrodes are not placed on the right position or that no signal is detected by the corresponding ECG electrodes, and, thus, the algorithm server 12 estimates that the quality of the ECG signal is low. In another embodiment, when the quality estimation algorithm is applied on each ECG signal, the algorithm server 12 detects whether there is a sharp slope on the ECG signal or not (block 402C). Referring to FIG. 6, detecting a sharp slop 60 appearing on the ECG signal indicates that the patient 15 moves violently, and, thus, the algorithm server 12 estimates that the quality of the ECG signal is low. In another embodiment, when the quality estimation algorithm is applied on each ECG signal, the algorithm server 12 detects whether there is a high-amplitude or high-frequency oscillation on the ECG signal or not (block 402D). Referring to FIG. 7, detecting high-amplitude or high-frequency oscillation 70 appearing on the ECG signal indicates that there is an electronic apparatus, such as a television, mobile phone, or a motor, near the sensor detector 10 or the patient 15 and then the ECG signal is interfered with the signals from the electronic apparatus, and, thus, the algorithm server 12 estimates that the quality of the ECG signal is low. In another embodiment, when the quality estimation algorithm is applied on each ECG signal, the algorithm server 12 detects whether there is a sharp baseline on the ECG signal or not (block 402E). Detecting a sharp baseline appearing on the ECG signal indicates that the lines of the corresponding ECG electrodes are pulled or moved by the patient 15, and, thus, the algorithm server 12 estimates that the quality of the ECG signal is low. In another embodiment, when the quality estimation algorithm is applied on each ECG signal, the algorithm server 12 detects whether there is any data loss for the ECG signal or not (block 402F). When the algorithm server 12 detects data loss for the ECG signal, the algorithm server 12 estimates that the quality of the ECG signal is low. In another embodiment, when the quality estimation algorithm is applied on each ECG signal, the algorithm server 12 detects whether there is any low voltage appearance for the whole ECG signal (block 402G). Detecting a low voltage appearance for the whole ECG signal indicates that the quality of the sensor device 10 or ECG electrodes is low, and, thus, the algorithm server 12 estimates that the quality of the ECG signal is low.

The above detection operations of the detection blocks 402A-402G are examples for quality estimation. The algorithm server 12 can selectively perform at least one of the detection operations of the detection blocks 402A-402G for accomplishing the quality estimation algorithm. In cases where the algorithm server 12 performs only one of the detection operations of the detection blocks 402A-402B for each ECG signal to estimate the quality, when the detection result of the performed detection block indicates that the quality of the ECG signal is low, the ECG signal is treated as an ECG signal with noise. In cases where the algorithm server 12 performs some or all of the detection operations of the detection blocks 402A˜402G for each ECG signal, when the number of detection results of the detection blocks which indicate that the quality of the ECG signal is low exceeds a threshold, the ECG signal is treated as an ECG signal with noise.

In an embodiment, one noise label is obtained for one ECG signal with low quality. For example, when the algorithm server 12 estimates that the quality of the ECG signal II is low, a noise label “LOW_QUALITY_II” is obtain for the ECG signal II. The noise label “LOW_QUALITY_II” will be shown on the label list on the display device 13. In an embodiment, for 12-lead ECG (including twelve ECG signals), when the number of ECG signals with low quality exceeds a predetermine value or when the quality of the specific ECG signal(s) is estimated to be low, a noise label is obtained for the twelve ECG signals. For example, when the number of ECG signals with low quality exceeds 4 or when the quality of the ECG signals I, III, and aVF is estimated to be low, a noise label “Low_Quality_ECG” is obtained for the twelve ECG signals. The noise label “Low_Quality_ECG” will be shown on the label list on the display device 13.

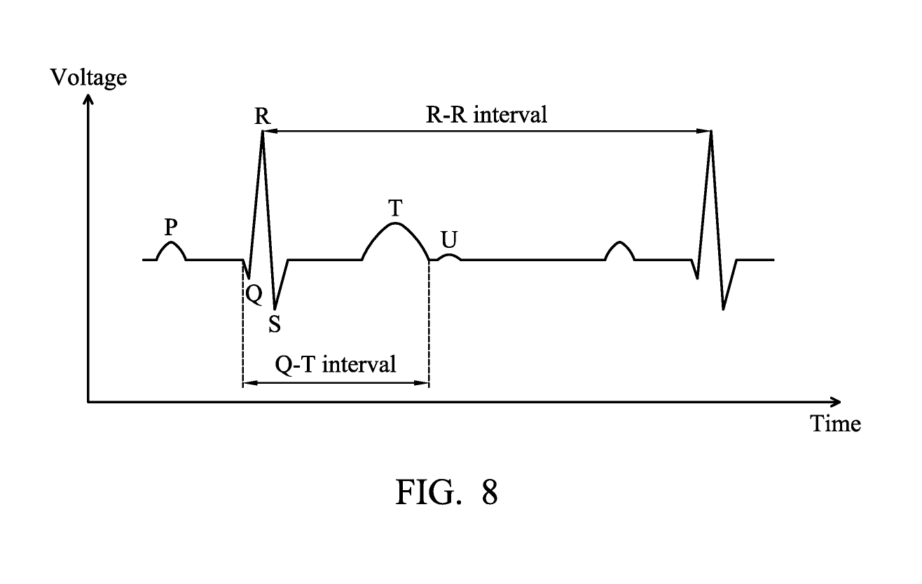

After receiving the ECG signals of the patient 15, the algorithm server 12 applies a feature extraction algorithm on the ECG signals (block 410). In an embodiment, the algorithm server 12 may detect beats of at least one ECG signal for obtaining the heart rate of the patient 15 (block 403). For example, as shown in FIG. 8, the algorithm server 12 detects the R-waves of the ECG signal I. Then, the algorithm server 12 applies a heart-rate algorithm to calculate the occurring frequency of the R-waves of the ECG signal I in a period of time to serve as the heart rate of the patient 15 (block 404). For example, the algorithm server 12 detects the R-waves of each of the twelve ECG signals. Then, the algorithm server 12 calculates occurring frequencies of the R-waves of the ECG signals in a period of time and calculates the average value or middle value of these occurring frequencies to serve as the heart rate of the patient 15.

In an embodiment, the algorithm server 12 may apply a waveform algorithm to extract ECG waveform features (block 405). For example, as shown in FIG. 8, for each ECG signal or at least one specific ECG signal, the algorithm server 12 extracts the waveform of the T-wave, the lowest level of the S-wave, and/or any other waveform feature which may be affected by cardiovascular diseases, such as the interval between the Q-wave and the T-wave (Q-T interval). The algorithm server 12 also extracts the interval of every two successive R-waves (R-R interval) of each ECG signal or at least one specific ECG signal. Each waveform feature may comprise multiple values. To make each feature more reliable, the proposed embodiment will calculate the middle value of the values of each waveform feature (block 406).

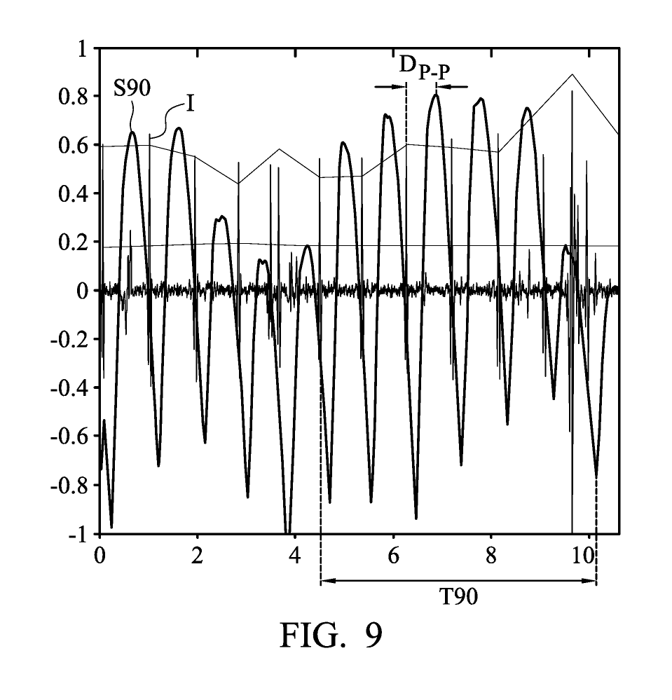

In some embodiments, the sensor device 10 detects the ECG signals and the vessel pulse signal of the patient 15 at the same time. A sensor of the sensor device 10 contacts a specific region, such as the right wrist of the patient 15. The sensor senses a vessel pulse waveform of the right wrist to generate the vessel pulse signal S11. The vessel pulse signal is also transmitted to the data server 11 for storage. When the algorithm server 12 issues the request RST to the data server 11, the data server 11 transmits the ECG signals and the vessel pulse signal to the algorithm server 12. Referring to FIG. 9, the algorithm server 12 calculates the time difference Dp-p between each peak of one ECG signal (such as the ECG signal I) and the peak of the vessel pulse signal S90 following the peak of the ECG signal to serve as a waveform feature and calculates the middle value of the values of the time difference Dp-p in in a period of time T90. The time difference Dp-p is referred to as a pulse transmission time (PTT) which indicates the time period when the pressure wave of the blood pressure is output to the right wrist from the heart. The pulse transmission time is an index for possible risk of arterial stiffness.

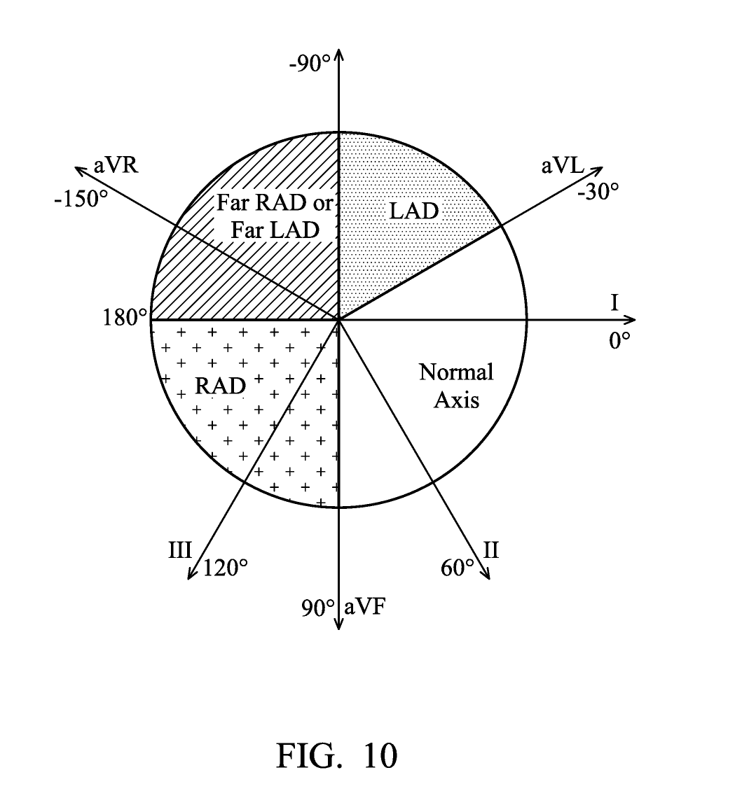

Moreover, the algorithm server 12 applies a heart-axis algorithm to determine the heart axis according to the ECG signals (block 407). When the algorithm server 12 averages all ECG signals, the direction of the average electrical depolarization can be indicated with an arrow (vector). The vector is the heart axis which is represented by a degree. A change of the heart axis or an extreme deviation can be an indication of pathology. Generally, a heart axis obtained from ECG signals of a healthy person is between −30° and 90° which is in the normal axis area shown in FIG. 10.

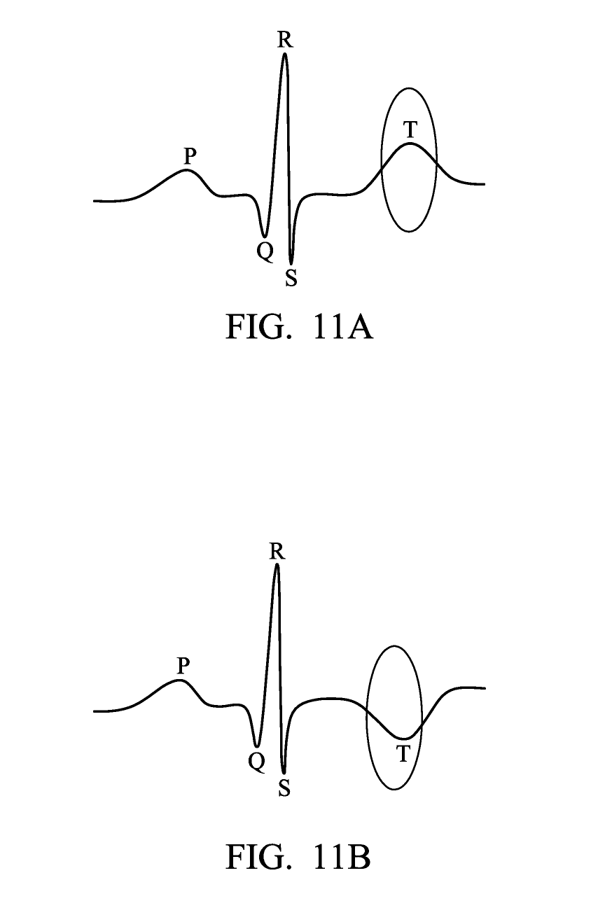

After the ECG waveform feature, the pulse transmission time, and the heart axis are obtained, the algorithm server 12 performs a labeling algorithm (block 408). In an embodiment, according to the labeling algorithm, the algorithm server 12 detects whether there is a T-wave inversion or not on each ECG signal or a specific ECG signal according to the extracted polarity of the T-wave (block 408A). For example, the algorithm server 12 detects whether there is a T-wave inversion or not on the ECG signal I according to the waveform of the T-wave. The waveform of the T-wave of the ECG signal I is one of the indexes for the possibility of myocardial infarction. Referring to FIG. 11A, the waveform of the T-wave of the normal ECG signal I of a healthy person is positive. When the waveform of the T-wave of the normal ECG signal I is negative, the algorithm server 12 detects that there is a T-wave inversion on the ECG signal I, shown in FIG. 11B, and gives an abnormal label “T-wave Inversion” or/and “Myocardial_Infarction” for the ECG signals of the patient 15.

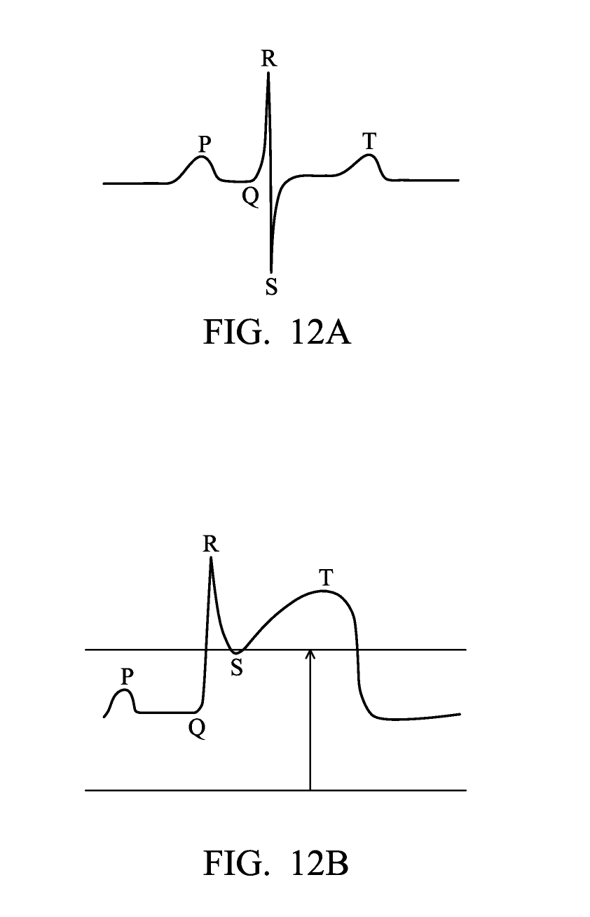

In an embodiment, according to the labeling algorithm, the algorithm server 12 detects that there is an ST elevation or not on each ECG signal or a specific ECG signal according to the extracted lowest level of the S-wave (block 408B). For example, the algorithm server 12 detects whether there is an ST elevation or not on the ECG signal I according to the extracted lowest level of the S-wave. The lowest level of the S-wave of the ECG signal I is one of the indexes for the possibility of myocardial injury. Referring to FIG. 12A, the lowest level of the S-wave of the ECG signal I of a healthy person is a negative level or lower than the lowest level of the Q-wave. When the lowest level of the S-wave of the ECG signal I is a positive level or higher than the lowest level of the Q-wave or when the lowest level of the S-wave of the ECG signal I is higher than a reference level or a historical level of the same wave which is obtained at the previous detection, shown in FIG. 12B, the algorithm server 12 detects that there is an ST elevation on the ECG signal I and gives an abnormal label “ST_Elevation” or/and “Myocardial_Injury” for the ECG signals of the patient 15.

In an embodiment, according to the labeling algorithm, the algorithm server 12 detects whether the patient 15 has hypertrophy according to the degree of the heart axis or not (block 408C). The degree of the heart axis being between 90° and 180° indicates that the patient 15 may suffer from hypertrophy. When the degree of the heart axis is between 90° and 180°, which is in the right axis deviation area (RAD) as shown in FIG. 10, the algorithm server 12 gives an abnormal label “Right Axis Deviation” for the ECG signals of the patient 15 and may further give an abnormal label “Hypertrophy”. In another embodiment, other cardiovascular diseases may induce the right axis deviation, such as a left posterior fascicular block, lateral myocardial infarction, right ventricular hypertrophy, and ventricular ectopy. Thus, when the degree of the heart axis is in the right axis deviation area (90°˜180°), the algorithm server 12 may further give at least one of the abnormal labels “Left_Posterior_Fascicular_block”, “Lateral_Myocardial_Infarction”, “Right_Ventricular_Hypertrophy”, and “Ventricular_Ectopy”.

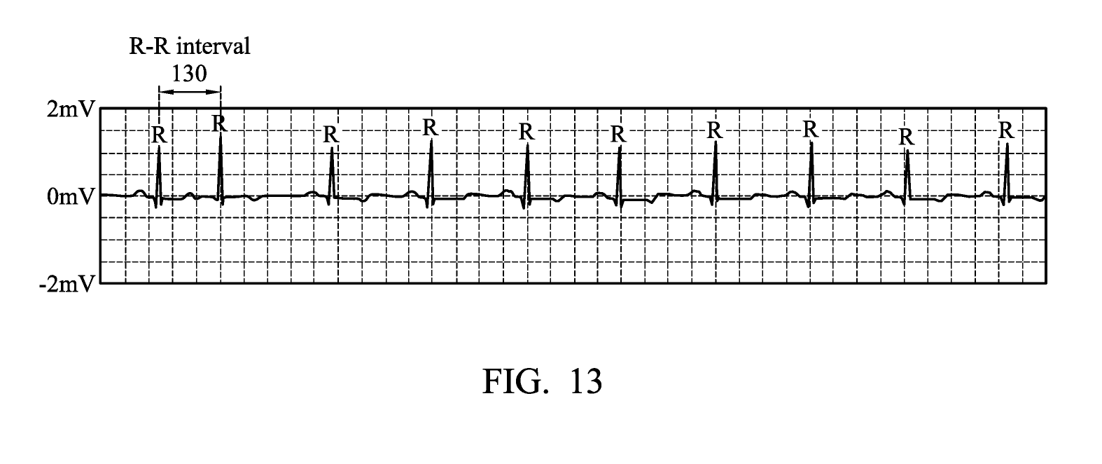

In an embodiment, according to the labeling algorithm, the algorithm server 12 detects whether there is arrhythmia or not according to the interval of every two successive R-waves (R-R interval) of each ECG signal or at least one specific ECG signal extracted in the block 405 (block 408D). The change of the R-R intervals of the ECG signals is one of the indexes for the possibility of arrhythmia. For example, the algorithm server 12 extracts the interval of every two successive R-waves (R-R interval) of the ECG signal I in the block 405 and then detects whether there is arrhythmia or not according to the extracted R-R intervals. Referring to FIG. 13, when the R-R intervals of the ECG signal I change in a period of time or when one of the R-R intervals of the ECG signal I (such as the R-R interval 130) is different from the others thereof, the algorithm server 12 detects that there is arrhythmia and gives an abnormal label “Arrhythmia” for the ECG signals of the patient 15.

Then, at the block 409, the algorithm server 12 generates the labeling result S12, which comprises the information of the labels in the blocks 402 and 408. The algorithm server 12 transmits the labeling result 12 to the display device 13 for displaying a label list. Accordingly, the labels obtained in the blocks 402 and 408 can be shown in the label list.

In the above embodiment, when the algorithm server 12 does not give any abnormal label in the block 48, a normal label “Normal_ECG” is obtained for the twelve ECG signals.

In the above embodiment, the labels comprise at least one noise label, at least one abnormal label, and a normal label. According to another embodiment, the labels can further comprise at least one labels related to the heart information, such as the heart rate and the heart axis. For example, in the block 403, the obtained heart rate is 74 bpm. The algorithm server 12 obtains a label “Heart_Rate” in the block 403. Accordingly, the information of the labeling result S12 further includes the label “Heart_Rate” and the information of the label “Heart_Rate” (that is 74 bpm). When the labeling result S12 is transmitted to the display device 13, the label “Heart_Rate” and the value “74 bpm” can also be shown on the label list. In an embodiment, after the algorithm server 12 determines the heart axis in the block 407, the algorithm server 12 also obtains a label “Heart_Axis” at the same block. For example, the determined heart axis is 50°. Accordingly, the information of the labeling result S12 further includes the label “Heart_Axis” and the information of the label “Heart Rate” (that is 50°). Through the transmission of the labeling result S12, the label “Heart_Axis” and the value “50°” can also be shown on the label list.

Moreover, the algorithm server 12 can also obtain an abnormal label related to the level of the heart rate. In the block 403, when the heart rate is obtained, the algorithm server 12 can determine whether the heart rate is higher than an upper threshold or whether the heart rate is lower than a lower threshold. When the algorithm server 12 determines that the heart rate is higher than the upper threshold, an abnormal label “Tachycardia” is provided for the ECG signals. When the algorithm server 12 determines that the heart rate is lower than the lower threshold, an abnormal label “Bradycardia” is provided for the ECG signals. Accordingly, the information of the labeling result S12 further includes the label related to the level of the heart rate. Through the transmission of the labeling result S12, the label related to the level of the heart rate can also be shown on the label list.

In an embodiment, the labels can further comprise a sleep stage label. It has been known that the heart rate of a human varies with the sleep stage. There are four sleep stages: an awake stage, a light sleep stage, a deep sleep stage, and a rapid eye movement sleep stage. In the block 403, when the heart rate is obtained, the algorithm server 12 can obtain a sleep stage label according to the heart rate. Thus, the sleep stage label can be a label “AWAKE” for the awake stage, a label “Light_sleep” for the light sleep stage, a label “Deep-Sleep” for the deep sleep stage, and a label “Rapid_eye_movement_Sleep” for the rapid eye movement sleep stage. Accordingly, the information of the labeling result S12 further includes the sleep stage label. Through the transmission of the labeling result S12, the sleep stage label can also be shown on the label list.

In the embodiment, the labeling algorithm can be performed by using a learning-based algorithm, such as a decision tree, a nearest neighbor algorithm, a support vector machine (SVM) algorithm, a random forest algorithm, an AdaBoost algorithm, a Naïve Bayes algorithm, a Bayesian-network, a neural network, a clustering algorithm, and a deep learning algorithm.

While the process flow described in FIG. 4 includes a number of operations that appear to occur in a specific order, it should be apparent that these processes can include more or fewer operations, which can be executed serially or in parallel, e.g., using parallel processors or a multi-threading environment.

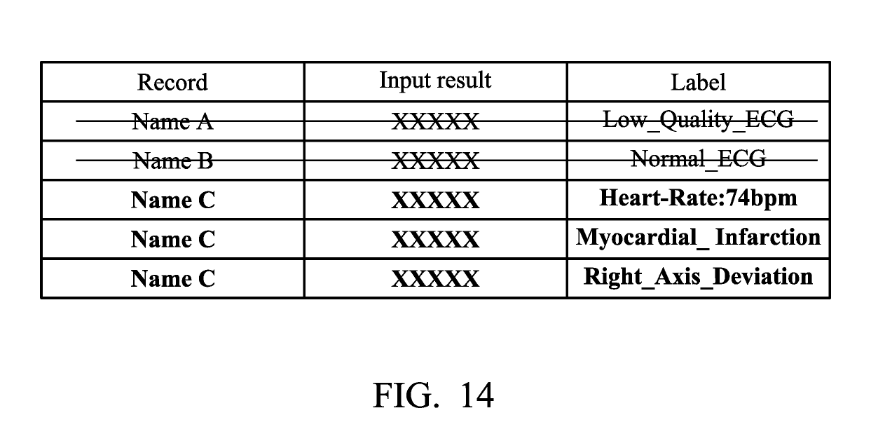

FIG. 14 shows an exemplary embodiment of the label list displayed on the display device 13. As shown in FIG. 14, the column “Record” lists the patient information, such as the patient's name, the column “Input Result” lists the information input by an interface of the display device 13, and the column “Label” lists the labels and the information of the labels. According to the above embodiments, the abnormal label is classified into the not-screened-out category, while the normal label and the noise label are classified into the screened-out category. In the label list, the display device 13 displays the labels classified into the not-screened-out category from the feature levels classified into the screened-out category by different formats or colors; for example, plain text, text with a marker, text with highlighted contrast, and text with lowlighted contrast. In order to distinguish the feature levels classified into the not-screened-out category from the labels classified into the screened-out category, the labels classified into the screened-out category are represented by text with lowlighted contrast or text with deleting lines. The contents of the label list is determined by the labeling result S12, and the information of the labeling result S12 is determined according to the results of the algorithms performed by the algorithm server 12.

In the above embodiment, the labels are obtained by the algorithm server 12 according to the extracted features of the ECG signals, such as the ECG waveform, the heart rate, the heart axis, and so on. In another embodiment, the display device 13 comprises an interface. A viewer, such as a doctor, can input a command through the interface to give a new label to the ECG signals or modify the original label.

While the invention has been described by way of example and in terms of the preferred embodiments, it is to be understood that the invention is not limited to the disclosed embodiments. On the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A healthcare system comprising:
a data server storing a plurality of physiological signals;
an algorithm server receiving the plurality of physiological signals from the data server, applying a plurality of algorithms on the plurality of physiological signals to obtain at least one feature of the plurality of physiological signals and generating a label according to the at least one feature;
a display device displaying the label; and
a communication network communicatively connecting the data server, the algorithm server, and the display device for providing signal transmission paths therebetween.

2. The healthcare system as claimed in claim 1, wherein the algorithm server classifies the label into a not-screened-out category or a screened-out category.

3. The healthcare system as claimed in claim 2, wherein the display device displays the label which is classified into a not-screened-out category or the label which is classified into a screened-out category by different formats or colors.

4. The healthcare system as claimed in claim 3, wherein formats comprise plain text, text with marker, text with highlighted contrast, and text with lowlighted contrast.

5. The healthcare system as claimed in claim 2, wherein the label is classified into the not-screened-out category when the label is an abnormal label.

6. The healthcare system as claimed in claim 5, wherein the abnormal label is an abnormal electrocardiography (ECG), a hypertrophy label, an arrhythmia label, a tachycardia label, a bradycardia label, or an ST elevation label.

7. The healthcare system as claimed in claim 2, wherein the label is classified into the screened-out category when the label is a normal label or a noise label.

8. The healthcare system as claimed in claim 2, wherein when the label is classified into the screened-out category, the algorithm server does not transmit the plurality of physiological signals to the display device.

9. The healthcare system as claimed in claim 1, wherein the plurality of physiological signals are obtained in response to electrocardiography, photoplethysmogram, motion, a body temperature, galvanic skin response, electroencephalograph, oxygen saturation, airflow in respiratory tract, a heart rate, pulse wave transit time, or blood pressure of an object.

10. The healthcare system as claimed in claim 1, wherein when the plurality of physiological signals are electrocardiography (ECG) signals of an object, the algorithm server applies the plurality of algorithms on the ECG signals to remove noise of the ECG signals, estimate quality of the ECG signals, detect a heart rate of the object, determine a heart axis, and extract predetermined features of the ECG signals and further applies a labeling algorithm to obtain the label according to at least one of the estimated quality, the detected heart rate, the heart axis, and the extracted predetermined features.

11. The healthcare system as claimed in claim 10, wherein the labeling algorithm comprises at least one of a decision tree, a nearest neighbor algorithm, a support vector machine (SVM) algorithm, a random forest algorithm, an AdaBoost algorithm, a Naïve Bayes algorithm, a Bayesian-network, a neural network, a clustering algorithm, and a deep learning algorithm.

12. The healthcare system as claimed in claim 1, wherein comprises an awake label, a light sleep label, a deep sleep label, or a rapid eye movement sleep label.

13. The healthcare system as claimed in claim 1, wherein the label is represented by a JSON format.

14. A monitoring method comprising:
obtaining a plurality of physiological signals;
applying a plurality of algorithms on the plurality of physiological signals to obtain at least one feature for the plurality of physiological signals;
generating a label according to the at least one feature; and
showing the label.

15. The monitoring method as claimed in claim 14 further comprising classifying each of the label into a not-screened-out category or a screened-out category.

16. The monitoring method as claimed in claim 15, wherein the label which is classified into a not-screened-out category or the label which is classified into a screened-out category is shown by different formats or colors.

17. The monitoring method as claimed in claim 16, wherein formats comprise plain text, text with maker, text with highlighted contrast, and text with lowlighted contrast.

18. The monitoring method as claimed in claim 15, wherein the label is classified into the not-screened-out category when the label is an abnormal label.

19. The monitoring method as claimed in claim 18, wherein the abnormal label is an abnormal electrocardiography (ECG), a hypertrophy label, an arrhythmia label, a tachycardia label, a bradycardia label, or an ST elevation label.

20. The monitoring method as claimed in claim 15, wherein the label is classified into the screened-out category when the label is a normal label or a noise label.
